(19) United States
(12) Patent Application Publication
(10) Pub. No.: US 2009/0275925 A1
(43) Pub. Date: Nov. 5, 2009

(54) DEVICE FOR THE CONTROLLED RELEASE OF A SUBSTANCE

(75) Inventors: Geert Langereis, Eindhoven (NL); Jaap Ruigrok, Asten (NL); Ventzeslav Petrov Iordanov, Valkenswaard (NL); Michel Paul Barbara Van Bruggen, Helmond (NL); Henrike Krijnsen, Boxtel (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/302,337

(22) PCT Filed: May 23, 2007
(86) PCT No.: PCT/IB07/51941

§ 371 Date: Nov. 25, 2008

(30) Foreign Application Priority Data
Jun. 2, 2006 (EP) 06114880.5

(51) Int. Cl. A61M 5/14; F16K 31/66

(52) U.S. Cl. 604/891.1; 29/890.132; 251/129.06

(57) ABSTRACT

An implantable drug delivery system is proposed which does not need internal electronics, but can be activated from outside the body. The system comprises a device (10) for the controlled release of a substance, the device (10) comprising at least one compartment (20) in a substrate (11), the compartment (20) being closed by a valve (30), wherein the valve is openable and comprises at least a first material (31) and a second material (32), the opening and/or closing of the valve (30) being provided upon heating and/or cooling of the first and/or second material (31, 32), the first material (31) being heated by electromagnetic induction.

The present invention relates to a device for the controlled release of a substance. The present invention further relates to a method of controllably releasing a substance from a compartment.

Accurate delivery of small, precise quantities of one or more chemicals into a carrier fluid is of great importance in many different fields of science and industry. Examples in medicine include the delivery of drugs to patients by means of intravenous methods, pulmonary or inhalation methods or by the release of drugs from vascular stent devices. Examples in diagnostics include the release of reactants into fluids used for DNA or genetic analysis, or combinatorial chemistry, or the detection of a specific molecule in an environmental sample. Other applications involving the delivery of chemicals into a carrier fluid include the release of fragrances and therapeutic aromas from devices into air and the release of flavouring agents into a liquid to produce beverage or food products.

Devices for the controlled release of a predefined quantity of a substance are generally known. For example, US patent application US 2004/0034332 A1 discloses an implantable device for controlled delivery of a drug, the device including a microchip which has reservoirs containing the molecules for release. The microchip device includes a substrate, at least two reservoirs in the substrate containing the molecules for release and a reservoir cap positioned on or within a portion of the reservoir and over the molecules, so that the molecules are controllably released from the device by diffusion through, or upon disintegration or rupture of, the reservoir caps. Each of the reservoirs of a single microchip can contain different molecules which can be released independently. One drawback of the known device is that they comprise an electronic control which requires electrical power, thereby introducing additional risks of malfunctioning.

It is therefore an object of the present invention to provide a device for the controlled release of a substance that provides higher reliability.

The above objective is accomplished by a device and a method for the controlled release of a substance according to the present invention, the device comprising at least one compartment in a substrate, the compartment being closed by a valve, and said valve being an openable valve. The valve comprises at least a first material and a second material, the opening and/or closing of the valve being controlled by heating and/or cooling of the first and/or second material, the first material being heated by electromagnetic induction.

An advantage of the device according to the invention is that the valve is a passive element and that it is possible to activate the valve from a remote point. This allows for example the implementation of a controlled substance or drug delivery system which does not require any power supply of its own. According to the prior art, batteries are used as an internal power supply, which involves a potential risk of failure. Whether the battery of an implanted device does still work or not cannot be detected. A further advantage of the device is that the implanted device can be smaller, especially not significantly larger, than the reservoir itself. Still a further advantage is that the manufacturing requirements and consequently the costs of the device are reduced.

A drug delivery system according to the present invention may be applied for the delivery of a single drug, but can be advantageously used in a system for the delivery of several different drugs from the same arrangement of a number of compartments or from the same device.

In a preferred embodiment of the invention, the first and/or second material is heated by a current induced in the first material by a varying electromagnetic field, the electromagnetic field being provided by a remote controller. Inductive heating of the first material is performed most reliably by applying the varying electromagnetic field, for example by supplying a coil with alternating current. Through electromagnetic induction a current will be induced in the first material, thus resulting in ohmic heating of the first material. The first and second material are preferably both conductive and arranged as a sandwich of thin plates inside a body. The magnetic field is preferably arranged perpendicularly to the plates and thus the second material plate is at least partially shielded by the first material. Both plates will have the same temperature almost instantly due to thermal conductivity of the first and second material.

According to the present invention, it is very much preferred that the valve is provided as a lid covering at least one compartment or an orifice leading to the compartment. Thereby, it is possible to achieve the advantages of the present invention by means of an arrangement which is very easy to manufacture. Especially, it is very easy to provide a lid as a release mechanism and to provide the first and second material as a part of the lid.

It is furthermore preferred that the release mechanism comprises at least a first material and a second material, the first material having a first thermal expansion coefficient, the second material having a second thermal expansion coefficient, and the first and second material being stacked. The stacked first and second material plates thereby allow to realise a very stable and robust device capable of being actuated by means of a change in temperature.

It is furthermore preferred that the valve opens upon the electromagnetic induction exceeding a threshold, the threshold being definable by choosing a combination of the first and second material of equal thickness and by a pre-defined stress in the stack of the first and second material, for example a bi-metal. The threshold depends upon the difference in thermal expansion coefficient and/or the summarised electrical conductivity of the first and second material. By setting a threshold for the opening of the valve, the device is secured against unwanted triggering. Further, a number of devices can be implanted, the valves of which open when the electromagnetic induction exceeds different thresholds for each device. This advantageously enables the devices to be either triggered sequentially or a certain number of devices to be triggered simultaneously. It is furthermore advantageous that, without induction, the valve is in its closed position. If the remote actuation is stopped for any reason, the release of the substance is immediately inhibited, which provides an advantageously high safety of the device.

In a preferred embodiment of the invention, the valve is reclosable and it is very much preferred that the release of the substance is acceleratable by a repeated opening and reclosing of the valve. In this manner the valve operates as a pumping device, which is advantageous if the substance is to be released quickly and/or in a high dose. It is further advantageous that the high amount of energy necessary for repeatedly opening and reclosing the valve is at hand, because according to the invention the power for opening and reclosing the valve is provided through electromagnetic induction by the remote controller outside the human or animal body and not by, for example, an internal battery.

It is furthermore preferred that the valve is movable between an open position and a closed position, the valve being stable in both the open and the closed position without the assistance of a force. The advantage of this embodiment is that the electromagnetic induction has to be applied only for the opening movement or for the reclosing movement. Once the valve is in either the open or closed position it will stay in that position even if the magnetic field is turned off.

In a further preferred embodiment of the invention, the device comprises a housing, the housing delimiting a space within which the valve is movable between a closed position and an open position. The advantage of this embodiment is that the pathway needed for the valve to open is kept free, for example if the device is implanted into human or animal tissue. The housing may also be used to restrict the opening of the valve to a certain extent, which provides the advantage of a free pathway for the valve and allows smaller devices.

In a still further preferred embodiment of the invention, the device comprises a link between the substrate and the valve to sealingly enclose the substance inside the compartment, the link being broken upon a movement of the valve relative to the substrate. The link is very advantageously provided to ensure a tight closure of the compartment before use. Consequently, between the manufacturing of the compartment and the use of the substance to be released, leaching out of the drug from the compartment is prevented.

The present invention further refers to a system comprising at least one device according to this invention and a remote controller, wherein the device is located inside a body and the remote controller is located outside the body. A body according to this invention is to be understood primarily in an anatomic sense but is applicable as well in a technical sense. It is an advantage of the system according to the invention that the device implanted into a human or animal body is controllable by a remote controller outside the body. The limitations regarding the special requirements of a device to be implanted in a human being or animal need not be met by the remote controller.

It is advantageously possible to enhance the safety and efficiency of the system by shifting dangerous and potentially unreliable functional elements to the remote controller. In a preferred embodiment, the device does not comprise any energy source or electronic component. Due to its simplicity, the implanted device is reliable and safe.

In a preferred embodiment, the remote controller comprises a coil supplied with alternating current, resulting in a fluctuating electromagnetic field, the electromagnetic field being essentially perpendicular to a surface of the body and/or being essentially directed towards the device. With the remote controller being located outside the body, a magnetic field of user-defined quantity and quality can be generated by a coil to which an alternating current is supplied.

In a further preferred embodiment of the system of the invention, opening and/or closing of the valve is provided at a temperature which is higher than the temperature of the body. It is very much preferred that the valve is inductively heated by the electromagnetic field to a temperature that is at least 10 degrees centigrade above the temperature of the body, more preferably at least 20 degrees centigrade above the temperature of the body. For a device implanted into a person or animal, unintentional opening of the valve due to for example hot weather conditions or pyrexia is prevented. The valve is enabled to stay closed even in the event of local and small temperature peaks. The compartments stay closed for example when transported or otherwise treated.

The present invention also includes a method of controllably releasing a substance from at least one compartment, using a device comprising at least one compartment in a substrate, the compartment being closed by a valve, said valve being openable, and said valve comprising at least a first material and a second material, and further using a remote control comprising a coil, the method comprising the steps of:
applying an alternating current to the coil, resulting in a fluctuating magnetic field directed towards the device,
heating the first material by inducing an electric current into the first material,
heating the second material,
opening of the valve upon heating the first and/or second material.

It is thereby possible to controllably release a substance in a highly reliable manner. Malfunction of the device, for example a power supply failure, is prevented.

The opening and/or closing of the valve upon heating and/or cooling is due to a difference in the thermal expansion coefficients of the first and second material. This provides for a reliable and quick opening and closing of the valve.

It is preferred to inductively increase the temperature of the first material by at least 10 degrees centigrade, more preferably by at least 20 degrees centigrade. Unintentional opening of the valve is thus prevented even in the event of local and small temperature peaks. The compartments stay closed for example when transported or otherwise treated.

The present invention also includes a method of manufacturing an inventive device comprising at least one compartment in a substrate, the compartment being closed by a valve arranged so as to be openable, the method comprising the steps of:
depositing or creating a first material and a second material on one side of the substrate for forming the valve,
etching the compartment into the substrate from another side until the first and/or second material is released,
filling the compartment with a substance,
applying a seal to close the compartment, and
preferably, depositing or creating a link between the substrate and the first and/or second material.

It is thereby possible to produce the inventive device in a very rapid and cost effective manner.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference numerals quoted below refer to the attached drawings.

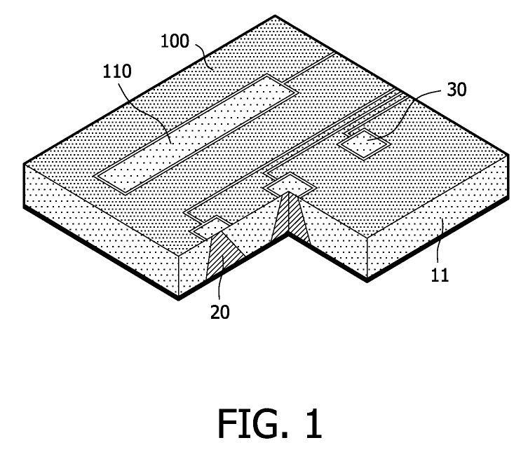

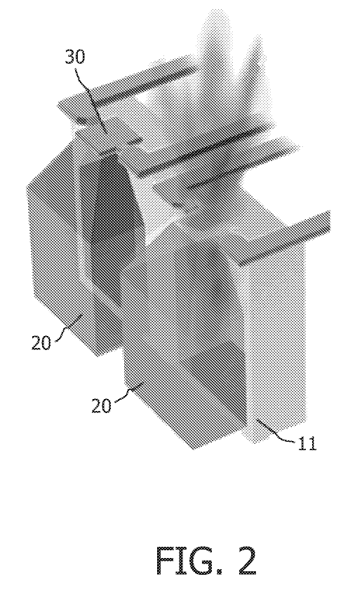

FIGS. 1 and 2 illustrate schematically a device 100 according to the prior art, showing in principle a structure of a prior art device.

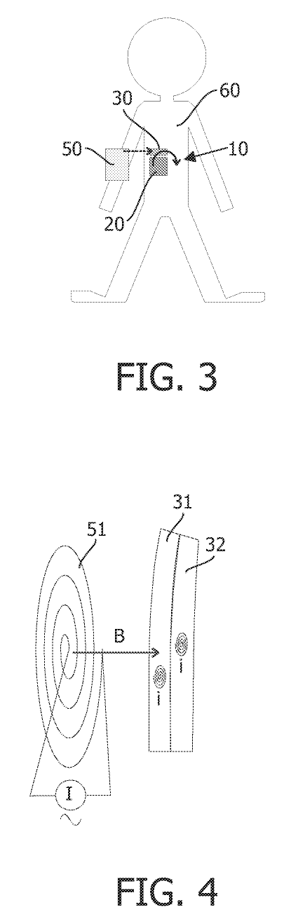

FIGS. 3 to 5 illustrate schematically an embodiment of a system according to the present invention.

FIG. 6 illustrates schematically a method of manufacturing a device according to the present invention.

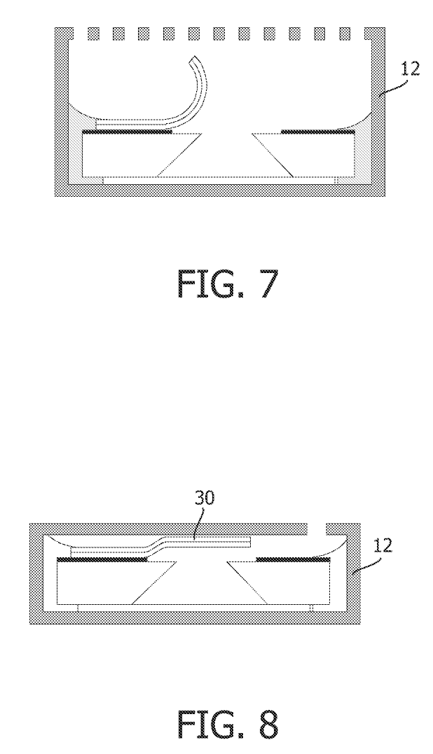

FIGS. 7 and 8 illustrate schematically two preferred embodiments of the device according to the present invention.

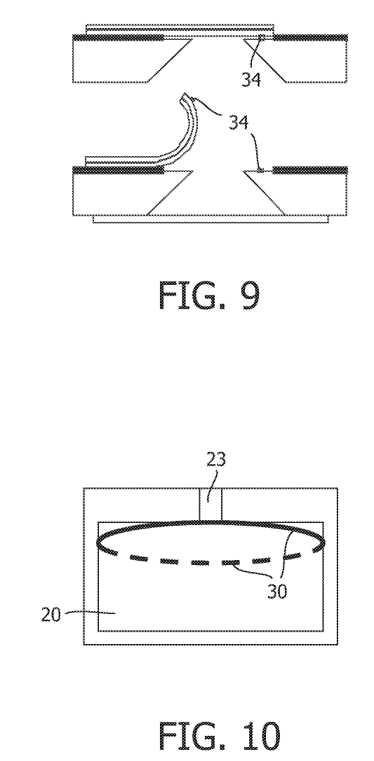

FIG. 9 illustrates schematically a further preferred embodiment of the device according to the present invention.

FIG. 10 illustrates schematically a preferred embodiment of the device according to the present invention with a bistable membrane.

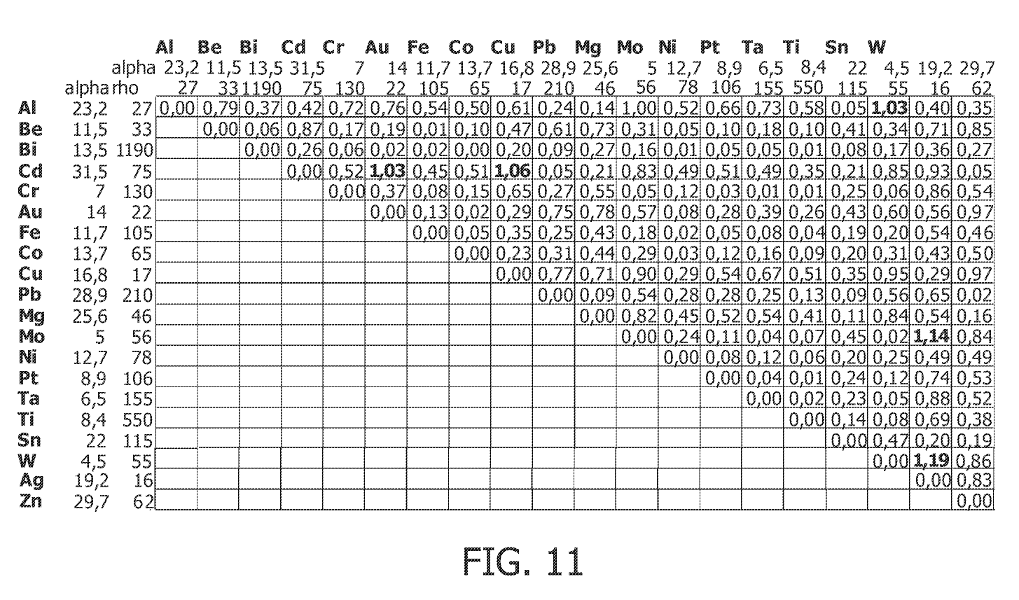

FIG. 11 shows a table of electrical resistivities and expansion coefficients of metals.

The present invention will be described with respect to particular embodiments and with reference to certain drawings, however, the invention is not limited thereto but only by the claims. The drawings described are only schematic and are non-limiting. In the drawings, the size of some of the elements may be exaggerated and not drawn to scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described of illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In FIGS. 1 and 2, a known device 100 according to the prior art is schematically shown. The known device 100 comprises a substrate 11 where a plurality of compartments 20 is located. The compartments 20 are closed by a valve 30, especially a closure cap 30. It can further be seen from FIG. 1 that there are electrode lines running to each of the compartments 20 or at least to or near to each of the valves 30. The connecting lines are not described with a reference sign in FIG. 1. The known device 100 further comprises an electrode area 110. FIG. 2 shows an intact release mechanism 30 and also an actuated valve 30 or closure cap 30 in order to disperse or release a substance, especially a drug.

In FIG. 3, a schematic overview of the inventive system is shown in connection with a human, represented by his outlined body 60. Implanted into the human body 60 is a device 10 for the controlled release of a substance, for example a drug. Device 10 comprises a compartment 20 which contains the substance, and a valve 30 by which the compartment 20 is closed. The valve 30 is openable and does not have a power supply of its own, but is activated from outside the body 60 by a remote control 50.

FIG. 4 shows schematically the interaction between the valve 30 and the remote control 50 in detail. The remote control 50 outside the body 60 comprises a coil 51, which is supplied with an alternating current, thus resulting in a fluctuating magnetic field. A magnetic field vector is represented by an arrow B, which is generally perpendicular to the surface of body 60, i.e. the skin of the human. The valve 30 of the device 10 implanted into the body 60 comprises a stacked sandwich of a first material 31 and a second material 32. Due to electromagnetic induction, eddy currents will be induced, at least into the front material 31. Due to this current, the first and/or second material 31, 32 heats up. The second material 32 may be shielded to a large extent from the electromagnetic field B and/or may be less sensitive to induction heating. Anyway, both materials 31, 32 will have the same temperature almost instantaneously due to thermal conductivity, and bending of the stack of the first and second material 31, 32 is initiated due to a difference in expansion coefficients. Inductive heating depends on the electrical conductivity of the material. The heating results in a difference in expansion between the first and second material 31, 32, thus causing bending of the valve 30.

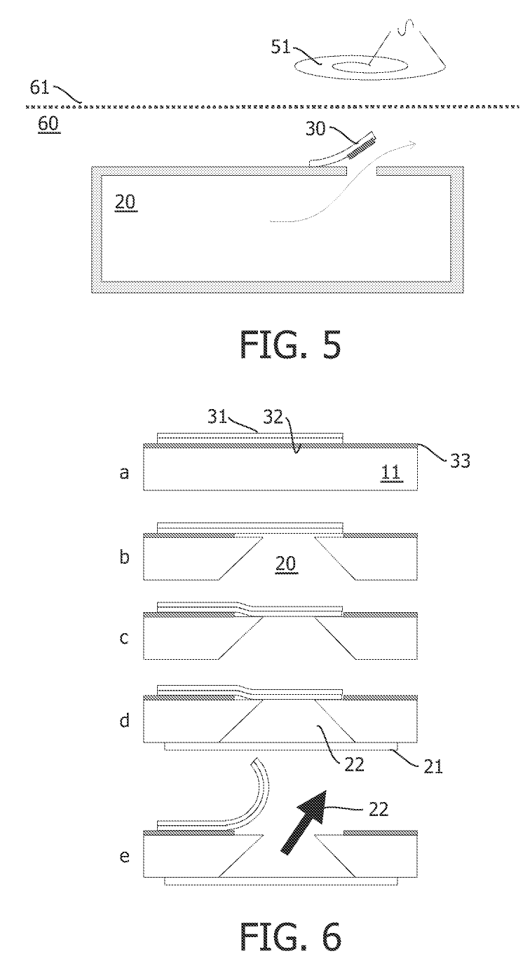

In FIG. 5, another detailed schematic view of the system is shown including the compartment 20 which comprises an orifice and which is implanted into the body 60. The dotted line 61 depicts the surface of the body 60. The valve 30 comprises the first material 31 and the second material 32, which bend upon inductive heating by coil 51 outside the body 60, so that the orifice is opened and the substance inside compartment 20 can evade.

In FIG. 6, a possible production process of the inventive device 10 is shown schematically by means of steps a-e. The reference signs for corresponding parts are not repeated for each step. Micro-electromechanical system methods, micro-moulding and micro-machining techniques known in the art can be used to fabricate the substrate 11 together with the compartment 20 from a variety of materials. Examples of suitable substrate materials include metals, ceramics, semiconductors, degradable and non-degradable polymers. Bio-compatibility of the substrate material typically is preferred for in-vitro device applications. The substrate, or portions thereof, may be coated, capsulated, or otherwise contained in a bio-compatible material before use. The substrate 11 can be flexible or rigid. In one embodiment, the substrate 11 serves as a support for a microchip device. In one example, the substrate 11 is made of silicon or another semiconductor material. The substrate 11 can have a variety of shapes for shaped surfaces. It can, for example, have a release side, i.e. an area having a valve 30, that is planar or curved. The substrate 11 may for example be in a shape selected from discs, cylinders, or spheres. In one embodiment, the release side can be shaped to conform to a curved tissue surface. This would be particularly advantageous for local delivery of a therapeutic agent to that tissue surface. In another embodiment, the backside (distal to the release side) is shaped to conform to an attachment surface. The substrate 11 may consist of only one material or may be made of a composite or multi-laminate material, that is, composed of several layers of the same or different substrate materials that are bonded together.

In step a, on the release side of the substrate 11, for example a silicon wafer 11, a stack is deposited consisting of a layer 33 of Silicon Nitride (LPCVD, low pressure chemical vapor deposition, Si₃N₄), the second material 32 and the first material 31.

Next, in step b, the silicon wafer 11 is etched from its backside by anisotropic etching in a Potassium Hydroxide (KOH) solution, thus providing the compartment 20. The Silicon Nitride layer 33 is etched from its backside (Buffered Oxide Etch, BOE/orthophosphoric acid, H₃PO₄) with sufficient over-etch to release the bi-metal stack of the first and second material 31, 32. Thereby, a small gap between the valve 30 or the lid 30 and the substrate 11 occurs, which might lead to a leaching out of the substance. To avoid this, the stack of materials should be chosen such that due to a stress difference in the different material layers, the lid 30 pushes downwards towards the substrate 11, which is shown in step c. Alternatively, a weak residual link 34 between the lid 30 and the substrate 11 can be provided (FIG. 9). Then the compartment 20 is filled from the backside with the substance 22 and a sealing layer 21 is applied, for example by laminating plastic foil as shown in step d, thereby completing the production process.

The compartment 20 is located in the substrate 11 along with the valve 30. The valve 30 is formed as a lid 30 covering one opening of the compartment 20 on the release side of the device 10. The lid 30 comprises the first material 31 and the second material 32. The two materials 31, 32 face each other along a contact surface. According to the present invention, the first material 31 has a different thermal expansion coefficient than the second material 32. Heating of the stack of the first and second material 31, 32 will result in bending of the stack of the first and the second material 31, 32. This allows for the realisation of a valve 30 or release mechanism at the opening of the compartment 20. When a temperature difference is applied to the closure lid 30, the bending of the first and the second material 31, 32 can be controlled and therefore opening or closing of the valve 30 of the compartment 20 can be performed. Inside the compartment 20 is located the substance, preferably a drug or another substance to be released in a very controlled manner. In step e, the valve 30 is depicted in its opened position so that the substance 22 can diffuse/flow outside.

In FIG. 7 an advantageous embodiment of the device 10 is depicted. After implantation of the device 10, it is necessary that the valve 30 has a free pathway in order to open the volume. Therefor the device 10 is packaged into a housing 12 which comprises openings through which the substance can flow from the device 10 to the outside. The valve 30 can be opened inside the housing 12 without being obstructed, for example by human tissue.

In FIG. 8 the embodiment of FIG. 7 is shown with an alternative housing 12 with a lower cover. The valve 30 will initially bend in a curved way but, due to the cover of the housing 12, will be stopped and remain in the position shown.

In FIG. 9 another advantageous embodiment of the device 10 is depicted. To avoid leaching out of the substance from the compartment 20, for example due to pressure variations, a weak link 34 is provided between the valve 30 and the substrate 11, as shown in the upper part of FIG. 9. In the lower part, the weak link 34, which is for example a ring of silicone nitride, is broken when the valve 30 is opened.

In FIG. 10, the valve 30 is a bi-stable membrane 30, which on the one hand is stable in its closed position, in which the membrane 30 is depicted as a full line, and which on the other hand is also stable in its lower, opened position, depicted as a dotted line. In the opening position of the membrane 30 the substance in the compartment 20 can flow outside through the orifice 23. The electromagnetic field, which is not depicted, is only used to cause the bi-stable membrane 30 to change from the closed position to the opened position and, if applicable, vice versa. Alternatively, the membrane 30 is not stable in its open position but drops back to its closed position when the actuating electromagnetic field is removed. The mechanical stability of the membrane 30 in one or both positions advantageously improves the on/off switching dynamic behaviour and the actuation temperature can be defined more exactly.

FIG. 11 shows a table of the electrical resistivity (rho in 10⁻⁹ Ohm m), which is the reciprocal of the electrical conductivity, and the linear expansion coefficient (alpha in 10⁻⁶ K⁻¹) for a number of metals, from which the first material 31 and the second material 32 may be chosen. The heating of the metal is the result of an induced electric current and depends therefor on the electrical conductivity of the metal. After heating, it is the difference in expansion that results in the deformation. The maximal bending of the bi-metal at a fixed induction voltage, determined by the given magnetic flux and frequency, assuming both layers of the first and second material 31, 32 are equally thick, is provided by a combination of first and second materials 31, 32 for a bi-metal valve 30 with maximum sum of conductivities and maximum difference in expansion coefficients. For all pairs of metals given in FIG. 1, the product of both values has been calculated. It appears that the best and most realistic combinations are Tungsten-Aluminum and Tungsten-Silver. The first and second materials 31, 32 and configuration should be chosen such that actuation takes place at temperatures significantly above the temperature of the body 60, because it is not acceptable that the valve opens due to fever or a hot day.

A formula to optimize the materials (=figure of merit) is

(σ₁+σ₂)(σ₁−σ₂)

where σ is the electrical conductivity and α the linear thermal expansion coefficient. To explain this (simplified) figure of merit, some assumptions have been made with respect to the configuration and response dynamics. It has to be noted that these assumptions are not essential for the invention, but they explain how to derive a useful figure of merit. In a realistic set-up, where design choices and behavior differs from the assumptions, the figure of merit still gives a performance indicator to optimize the material choices.

It is assumed that both materials 31 and 32 have the same thickness. In addition, it is assumed that the layers are so thin that bending is the result of changes in length only, and not of differences in elasticity.

Due to the geometry in the proposed configuration, the induction voltage is not dependent on the choice of materials. The induced power is proportional to the squared induction voltage, divided by the electrical resistance. So:

$$Power ∝ \frac{{V}_{ind}^{2}}{R} = {V}_{ind}^{2} {σ}_{total} ∝ {V}_{ind}^{2} ( {σ}_{1} + {σ}_{2} )$$

It is assumed for simplicity that the valve is only heated for such a short time that there is no leakage of heat to the environment. In that case the increase in temperature of the bi-metal is proportional to the power (given above) times the heating time, and inversely proportional to the total heat capacity of the bi-metal. The total heat capacity is proportional to

Cₜₒₜₐₗ∝cᵥ₁+cᵥ₂∝r₁c_{g}_{₁}+r₂c_{g}_{₂}

where r is the density and c the specific heat capacitance. This is a simplified formula because it is again assumed that the two layers are equal in thickness. The curvature (bending) is proportional to the difference in length, i.e. proportional to

ΔT·(β₁−α₁).

Since, due to the short heating time, the temperature rise is proportional to the power and inversely proportional to the specific heat capacity cᵥ, the bending at the given induction voltage becomes proportional to

$$\frac{( {σ}_{1} + {σ}_{2} ) \cdot ( {α}_{1} - {α}_{1} )}{{c}_{v 1} + {c}_{v 2}}$$

In the simplified figure of merit, (σ₁+σ₂)(α₁−α₂), the denominator cᵥ₁+cᵥ₂ is not considered, because heat capacities per unit of volume cᵥ of different materials do not differ so much in practice (since the size of atoms and their degrees of freedom do not differ very much from metal to metal). Hence, the heat conductances and the difference in the linear expansion coefficients of the metals used in the bi-metal generally determine the bending performance of a certain combination of metals.

1. Device (10) for the controlled release of a substance, the device (10) comprising at least one compartment (20) in a substrate (11), the compartment (20) being closed by a valve (30), wherein the valve is openable and comprises at least a first material (31) and a second material (32), the opening and/or closing of the valve (30) being provided upon heating and/or cooling of the first and/or second material (31, 32), the first material (31) being heated by electromagnetic induction.

2. Device (10) according to claim 1, wherein the first and/or second material (31, 32) is heated by a current induced in the first material (31) by a varying electromagnetic field, the electromagnetic field being provided by a remote controller (50).

3. Device (10) according to claim 1, wherein the first material (31) has a first thermal expansion coefficient (41) and the second material (32) has a second thermal expansion coefficient (42), the first and second material being stacked.

4. Device (10) according to claim 1, wherein the valve opens upon the electromagnetic induction exceeding a threshold, the threshold being definable by choosing a combination of the first and second material (31, 32), depending upon the difference in thermal expansion coefficient (41, 42) and/or the summarized electrical conductivity of the first and second material (31, 32).

5. Device (10) according to claim 1, wherein the valve (30) is reclosable and the release of the substance is acceleratable by a repeated opening and reclosing of the valve.

6. Device (10) according to claim 1, wherein the valve (30) is movable between an open position and a closed position, the valve being stable in both the open and the closed position without the assistance of a force.

7. Device (10) according to claim 1, wherein the valve (30) is provided as a lid (30) covering at least one compartment (20) or an orifice leading to the compartment.

8. Device (10) according to claim 1, further comprising a housing, the housing delimiting a space within which the valve (30) is movable between a closed position and an open position.

9. Device (10) according to claim 1, further comprising a link (36) between the substrate and the valve for sealingly enclosing the substance inside the compartment (20), the link being broken upon movement of the valve (30) relative to the substrate (11).

10. System comprising at least one device (10) according to claim 1 and a remote controller, wherein the device is located inside a body (60) and the remote controller (50) is located outside the body.

11. System according to claim 10, wherein the device (10) does not comprise any energy source or electronic component.

12. System according to claim 10, wherein the remote controller comprises a coil supplied with an alternating current, resulting in a fluctuating magnetic field, the magnetic field being essentially perpendicular to a surface of the body and/or being essentially directed towards the device (10).

13. System according to claim 10, wherein the opening and/or closing of the valve (30) is provided at a temperature which is higher than the temperature of the body (60).

14. System according to claim 12, wherein the valve (30) is inductively heated by the magnetic field to a temperature that is at least 10 degrees centigrade above the temperature of the body (60).

15. Method of controllably releasing a substance from at least one compartment (20), using a device (10) comprising at least one compartment (20) in a substrate (11), the compartment (20) being closed by a valve (30), wherein said valve (30) is openable, and said valve (30) comprises at least a first material (31) and a second material (32), and
further using a remote control comprising a coil, the method comprising the steps of:
applying an alternating current to the coil, resulting in a fluctuating magnetic field directed towards the device (10),
heating the first material (31) by inducing an electric current into the first material,
heating the second material (32),
opening of the valve (30) upon heating of the first and/or second material.

16. Method according to claim 15, wherein the opening and/or closing of the valve (30) upon heating and/or cooling is due to a difference in the thermal expansion coefficients (41, 42).

17. Method according to claim 15, wherein the difference in temperature inductively applied to the first material (31) is at least 10 degrees centigrade.

18. Method according to claim 15, wherein the difference in temperature inductively applied to the first material (31) is at least 20 degrees centigrade.

19. Method of manufacturing a device (10) comprising at least one compartment (20) in a substrate (11), the compartment (20) being closed by a valve (30), wherein the valve (30) is arranged so as to be openable, and the method comprises the steps of:
depositing or creating a first material (31) and a second material (32) on one side of the substrate (11) for forming the valve (30),
etching the compartment (20) into the substrate (11) from another side until the first and/or second material is released,
filling the compartment with a substance and
applying a seal to close the compartment.

20. Method according to claim 19, further comprising the step of depositing or creating a link between the substrate and the first and/or second material.
